(19) United States
(12) Patent Application Publication
(10) Pub. No.: US 2011/0200647 A1
(43) Pub. Date: Aug. 18, 2011

(54) PULMONARY DISEASE TREATMENT

(75) Inventors: Francis M. Cuss, Bernardsville, NJ (US); Keith B. Nolop, Redwood City, CA (US); Ulo A. Palm, Scotch Plains, NJ (US); Heribert W. Staudinger, Green Brook, NJ (US)

(73) Assignee: Schering Corporation, Kenilworth, NJ (US)

(21) Appl. No.: 13/093,307

(22) Filed: Apr. 25, 2011

Related U.S. Application Data
(63) Continuation of application No. 11/850,509, filed on Sep. 5, 2007, now abandoned.
Application No. 11/850,509 is a continuation of application No. 10/948,987, filed on Sep. 24, 2004, now abandoned.
(60) Provisional application No. 60/551,596, filed on Mar. 9, 2004.
(60) Provisional application No. 60/506,468, filed on Sep. 26, 2003.

(51) Int. Cl. A61K 9/14; A61K 31/58; A61P 11/00

(52) U.S. Cl. 424/400; 514/172

(57) ABSTRACT

The invention relates to treating chronic obstructive pulmonary disease, involving the administration by inhalation of mometasone furoate particles in daily doses where at least about 250 μg of the inhaled particles have sizes equal to or less than 6.5 μm.

This application claims benefit of priority to U.S. Provisional Patent Application Ser. Nos. 60/506,468 filed Sep. 26, 2003 and No. 60/551,596 that was filed on Mar. 9, 2004.

### INTRODUCTION TO THE INVENTION

The present invention relates to a treatment of diseases of the pulmonary system, particularly obstructive pulmonary diseases, using an inhaled corticosteroid drug.

Chronic obstructive pulmonary disease (“COPD”) is a medical condition that does not have a precise definition, but is generally considered to include one or both of chronic bronchitis and emphysema. Chronic bronchitis is characterized by a persistent (such as more than one year) productive cough that is not due to a medically defined cause such as a microbial infection or carcinoma. Emphysema is an abnormal permanent non-uniform enlargement of air spaces distal to the terminal bronchioles, including destruction of the walls of the air spaces.

COPD is not considered to include the allergic condition asthma, in which the airway restriction is reversible upon administration of a bronchodilating drug; however, it is possible for a patient to have COPD together with asthma. COPD also does not include certain other diagnosable conditions such as bronchiectasis, cystic fibrosis, or obliterative bronchiolitis.

COPD is a serious medical problem, as it is a progressive disease that cannot be cured and is a leading cause of death. A large fraction of the population suffers to some degree from the disorder, and causation factors are thought to include tobacco smoke, exposure to chemical fumes during employment, air pollution, and others. It is perceived that the incidence of the disease is increasing rapidly. The customary COPD treatment includes administering a bronchodilator and, if an adequate symptomatic response is not obtained, adding an anticholinergic agent and/or theophylline to the therapy. Oral steroid drugs are frequently also administered to produce systemic concentrations for treating severe symptoms, but the serious such drugs is very well known. The goal of current COPD therapy is to limit progression of the disease, and to minimize the number of serious exacerbations.

In asthma, the airway is characterized by an eosinophilic inflammation, while the airway in COPD is characterized by a presence of neutrophils; the prevailing current view is that asthmatic inflammation is markedly suppressed by corticosteroids, while these drugs have no appreciable effect on inflammation in COPD (see P. J. Barnes, “Mechanisms in COPD: Differences from Asthma,” Chest, Vol. 117, February 2000 Supplement, pages 10S-14S). A review of recent studies that were conducted to determine the effects of inhaled corticosteroids in COPD has been reported by A. Alsaeedi et al., “The Effects of Inhaled Corticosteroids in Chronic Obstructive Pulmonary Disease: A Systematic Review of Randomized Placebo-Controlled Trials,” American Journal of Medicine, Vol. 113, pages 59-65 (2002), who concluded that patients treated with the drugs generally had a reduced rate of exacerbations, but there was no effect shown on patient mortality. The specific inhaled steroid drugs that were used in the studies reviewed by Alsaeedi et al. were budesonide, fluticasone, triamcinolone and beclomethasone.

G. Ferguson, “Recommendations for the Management of COPD,” Chest, Vol. 117, February 2000 Supplement, pages 23S-28S, compared the treatment guidelines that had been published by the European Respiratory Society, the British Thoracic Society and the American Thoracic Society. Inhaled corticosteroids are considered to possibly be of value for COPD by the European and British societies, but the American society does not recommend their use.

Mometasone furoate is a corticosteroid drug having anti-inflammatory properties, which has been used for several years in dermatological products, including ointment, lotion, and cream forms. It has the chemical name 9a,21-Dichloro-11β,17-dihydroxy-16α-methylpregna-1,4-diene-3,20-dione 17-(2-furoate), an empirical formula C₂₇H₃₀Cl₂O₆, and a molecular weight of 521.44. The drug has been proposed for use in inhalation forms for the treatment of disorders such as asthma, as mentioned in the following U.S. Pat. Nos. 5,474,759; 5,889,015; 6,057,307; 6,068,832; 6,365,581; 6,503,482; 6,677,322; and 6,677,323.

### SUMMARY OF THE INVENTION

The invention encompasses a method for treating chronic obstructive pulmonary disease, wherein there are administered by oral inhalation particles of mometasone furoate in daily doses where at least about 250 μg of the particles have sizes equal to or less than about 6.5 μm. Preferably, no more than about ten percent of particles in the daily dose have sizes less than about 1 μm. Daily doses will be administered once per day, preferably in the evening, or in two divided, and preferably equal, doses at approximately twelve-hour intervals.

In a preferred embodiment of the present invention, the total daily dosage of mometasone furoate is 800 μg, preferably administered once daily, preferably in the evening. This preferred embodiment of the present invention is sometimes referred to as “800 μg QDPM”.

In another preferred embodiment, the total daily dosage of mometasone furoate is 800 μg and is administered in a single dose to a patient who has a history of repeated COPD exacerbations, and the administration of the 800 μg single dose is preferably in the evening.

### BRIEF DESCRIPTION OF THE DRAWINGS

FIG. 1 is a graph showing results from the study of Example 1.

FIG. 2 is a graph showing results from the study of Example 2.

### DETAILED DESCRIPTION

The invention includes a therapy for chronic obstructive pulmonary disease comprising the administration by oral inhalation of mometasone furoate particles. It has been found that progression of the disease state can be markedly reduced when there are inhaled at least about 250 μg per day of mometasone furoate particles having sizes not exceeding about 6.5 μm, in a single daily dose or in divided, approximately equal doses at approximately twelve-hour intervals. To minimize a patient's systemic exposure to the drug, it is advisable to restrict the amount of inhaled mometasone furoate particles having sizes less than about 1 μm to constitute less than about ten percent by weight of the total particles measuring no more than about 6.5 μm. The activity of mometasone furoate is essentially local, at the points where particles are deposited in the respiratory tract.

In general, a minimum effective amount of drug will be administered. That amount will frequently not exceed about 600 μg per day of inhaled mometasone furoate particles having sizes equal to or less than about 6.5 μm.

Preferably, the daily dose of mometasone furoate contains at least about 200 μg of drug particles having diameters less than about 4.4 μm, at least about 175 μg of drug particles having diameters less than about 3.3 μm, at least about 75 μg of drug particles having diameters less than about 2 μm, and no more than about 50 μg of particles having diameters less than about 1 μm.

The mometasone furoate particles for inhalation can be provided by any of a number of device and composition combinations. Pressurized metered dose inhalers containing mometasone furoate particles and a liquefied low-boiling and substantially inert propellant, such as a hydrofluoroalkane (e.g., 1-1-1,2-tetrafluoroethane or 1,1,1,2,3,3,3-heptafluoropropane), are suitable; examples of compositions that can be delivered by a metered dose inhaler are given by Berry et al. in U.S. Pat. No. 6,068,832. Also useful are aqueous suspensions of mometasone furoate monohydrate particles, such as are described in U.S. Pat. No. 6,187,765 to Harris et al.; such aqueous suspensions will be aerosolized for inhalation using a nebulizer device that generates the aerosol by ultrasonic means or with a compressed gas, and many of these devices are commercially available. A very convenient inhalation source of mometasone furoate particles is a dry powder inhaler, including devices that contain a unit dose of the drug in a capsule which is opened inside the device immediately prior to an inhalation (one example of which is shown in U.S. Pat. No. 3,991,761 to Cocozza), or devices that contain stored multiple unit doses of the drug and meter an appropriate amount before each inhalation.

Particularly useful in the practice of the present invention is the multiple-dose mometasone furoate dry powder inhaler being sold by business units of Schering-Plough Corporation under the trademark “ASMANEX TWISTHALER.” The features and operation of this inhaler are described in U.S. Pat. No. 6,240,918 to Ambrosio at al. A suitable composition which is contained within the inhaler is described in U.S. Pat. No. 6,503,537 to Yang, and comprises relatively hard agglomerates of lactose and mometasone furoate particles wherein the weight ratio of lactose to drug is about 5.8:1. Particle sizes of the drug substance should be adjusted, before agglomerates are formed, such that the inhaler will deliver proper amounts of inhalable mometasone furoate particles meeting the size requirements for the therapy of this invention; device design and agglomerate to formulation parameters for achieving this result are discussed in the Ambrosio et al. and Yang patents mentioned in this paragraph.

Dosage ranges for mometasone furoate are discussed, e.g., in U.S. Pat. Nos. 6,365,581 and 6,057,307. (See, e.g, U.S. Pat. No. 6,057,307 at col. 6, line 45-col. 7, line 20, expressly incorporated herein by reference). In a preferred is embodiment of the present invention, the total daily dosage of mometasone furoate is 800 μg, preferably administered once daily, preferably in the evening (800 μg QDPM).

In another preferred embodiment, the total daily dosage of mometasone furoate is 800 μg and is administered in a single dose to a patient who has a history of repeated COPD exacerbations, and the administration of the 800 μg single dose is preferably in the evening. For information on exacerbations of COPD and identification of disease severity for COPD patients, reference can be made, e.g., to Global Initiative for Chronic Obstructive Lung Disease: Global Strategy for the Diagnosis, Management, and Prevention of Chronic Obstructive Lung Disease (NHLB/WHO Workshop Report, Bethesda, Md., National Heart, Lung and Blood Institute, April 2001; NIH Publication No. 2701:1-100), see, e.g., pages 39-40, 55, and 87-94 of the 2003 Update (GOLD website: www.goldcopd.com). The severity of the disease state in a patient can be quantified by objective pulmonary function tests, including a measurement of the patient's forced expiratory volume in 1 second (FEV₁). When this result is about 65 to 79 percent of the predicted value (determined using a formula that takes into account the patient's age and size), the airway obstruction is considered to be mild. For an FEV₁ value about 50 to 64 percent of predicted, the airway obstruction is classified as moderate; if the value is less than 50 percent of predicted, the airway obstruction is considered to be severe; and if the value is less than 30 percent the airway obstruction is considered to be very severe. This test utilizes relatively simple and inexpensive equipment, and therefore is widely used for disease state diagnosis, and to monitor the progression of COPD and other lung and airway disorders during treatment.

Other COPD therapy is typically useful, in addition to the administration of inhaled mometasone furoate particles according to this invention. This will include to the usual treatments with one or more agents such as bronchodilators, anticholinergic agents, theophylline, and others as needed to limit exacerbations of the disease state. It is an advantage of the therapy of this invention that the concomitant use of orally administered corticosteroids can frequently be greatly reduced or eliminated, to minimize a patient's systemic exposure to these drugs.

### Example 1

A randomized, double-blind crossover clinical trial was conducted to compare the effects of orally inhaled mometasone furoate and a placebo, in patients suffering from chronic obstructive pulmonary disease. The drug was delivered from a multiple-dose dry powder inhaler charged with a mixture of mometasone furoate and lactose (having a component weight ratio of 1:5.8) in an agglomerated form. Placebo inhaler units contained only the lactose powder. The drug-containing inhalers delivered the following mean amounts of mometasone furoate particles per inhalation, as measured using an Anderson Cascade Impactor (from Thermo Anderson, Smyrna, Ga. U.S.A.) at an air flow rate through the inhaler of 60 L/minute:

| μg Mometasone Furoate | Particle Size, μm |
|---|---|
| 147 | <8.6 |
| 142 | <6.5 |
| 123 | <4.4 |
| 95 | <3.3 |
| 42 | <2.0 |
| 14 | <1.1 |

Evaluable patients participating in the study numbered 578, all having been diagnosed with COPD of moderate severity, being at least 40 years of age, and also being maintained for at least three months prior to commencement of the study on daily inhalations of a corticosteroid drug (even though there was no regulatory agency-approved COPD therapy using an inhaled corticosteroid), Each patient demonstrated a resistance to FEV₁ reversibility (i.e., a low FEV₁ reversibility) with the inhaled bronchodilator salbutamol (albuterol), a criterion for inclusion in the study being a reversibility after salbutamol administration to amounting to less than ten percent of the patient's predicted FEV₁ value; this minimized the confounding influence of asthma co-morbidity. The patients were enrolled at a total of 35 sites in 24 countries, and were prescribed two inhalations from the supplied inhaler device each evening for a twelve-month period, with evaluations by a physician after 1, 3, 6, 9, and 12 months of participation. A total of 294 of the patients were randomized to receive inhalers containing the drug, while 284 patients received placebo inhalers. All of the patients were permitted to further use inhaled salbutamol as needed.

Results of the study were as follows, where the ΔFEV₁ value represents the average of differences between the patients' FEV₁ scores at the beginning of the study and the FEV₁ scores at the time of the subsequent measurement, with all of the FEV₁ testing being performed after administration of inhaled salbutamol. For each time point, the number of patients remaining in the study is given.

| Treatment | No. Patients | Months | ΔFEV₁ (mL) |
|---|---|---|---|
| Placebo | 261 | 1 | −18 |
| ″ | 216 | 3 | −27 |
| ″ | 179 | 6 | −29 |
| ″ | 162 | 9 | −21 |
| ″ | 153 | 12 | −28 |
| Drug | 276 | 1 | 5 |
| ″ | 239 | 3 | 9 |
| ″ | 225 | 6 | −2 |
| ″ | 195 | 9 | 22 |
| ″ | 192 | 12 | 7 |

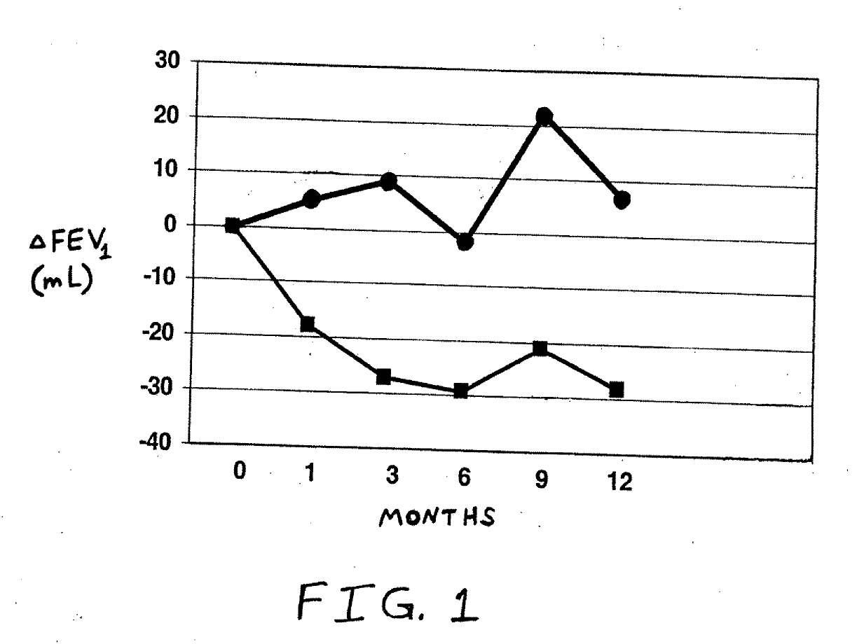

These results are summarized in the graph of FIG. 1, where data points for mometasone furoate are shown by round symbols () and placebo data points are shown by square symbols (▪). From this graph, it is apparent that those patients using the placebo inhalers generally declined in pulmonary function over the trial period, while the patients inhaling mometasone furoate particles generally maintained or slightly improved their pulmonary function.

From a statistical analysis of the data, it was determined that the mean change from baseline over the twelve-month period was an increase in ΔFEV₁ of 4 mL for patients receiving the mometasone furoate, and a decrease of 36 mL for placebo patients. This difference between treatments is statistically significant, using a longitudinal analysis of variance model that includes sources of variation due to smoking status, previous dosing of inhaled corticosteroid, treatment, time, time-by-treatment interaction, initial FEV₁ level and the rate of change in FEV₁ level.

### Example 2

A randomized double-blind, parallel-group clinical trial was conducted to compare the effects of orally inhaled mometasone furoate and a placebo, in patients suffering from chronic obstructive pulmonary disease. The multiple dose dry powder inhaler and placebo inhaler described in the preceding example were used for this study.

A total of 769 patients met the criteria for evaluability, from 95 sites in 11 countries. The patients had not previously been treated with corticosteroids, either inhaled or orally dosed, but otherwise met the criteria of the preceding example. In the study, patients were prescribed either two inhalations from the inhaler each evening, or one inhalation each morning and one inhalation each evening, with a twelve-month treatment duration.

Results of the study were as follows, where the ΔFEV₁ value represents the average of differences between the patients' FEV₁ scores at the beginning of the study and the FEV₁ scores at the time of the subsequent measurement, with all of the FEV₁ testing being performed after administration of inhaled salbutamol. For each time point, the number of patients remaining in the study is given.

| | Placebo | | Once Daily | | Twice Daily | |
|---|---|---|---|---|---|---|
| | No. | ΔFEV₁ | No. | ΔFEV₁ | No. | ΔFEV₁ |
| Months | Patients | (mL) | Patients | (mL) | Patients | (mL) |
| 1 | 248 | 6 | 257 | 66 | 263 | 72 |
| 3 | 218 | −9 | 239 | 57 | 241 | 79 |
| 6 | 189 | −29 | 217 | 44 | 209 | 36 |
| 9 | 158 | −37 | 185 | 37 | 198 | 58 |
| 12 | 156 | −38 | 190 | 38 | 191 | 31 |

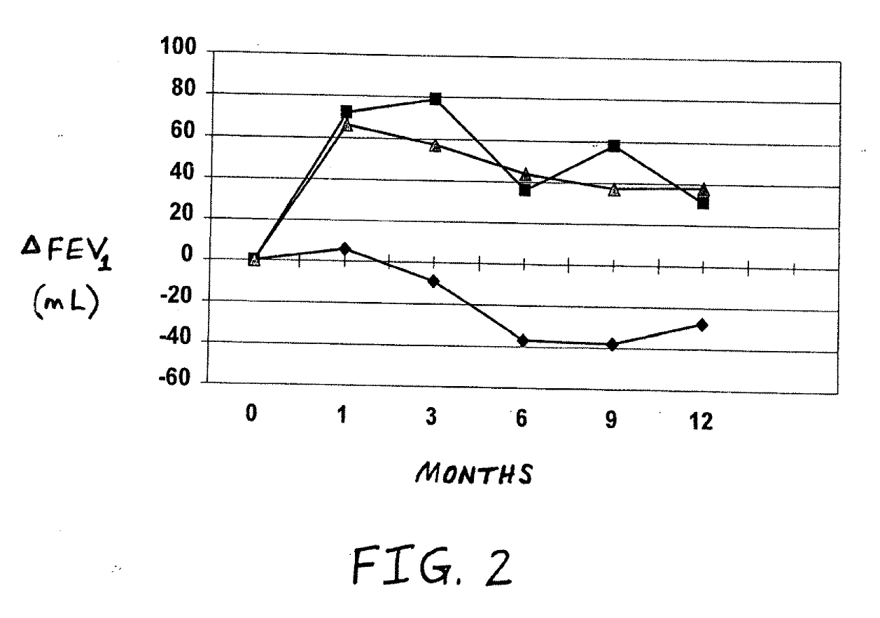

These results are summarized in the graph of FIG. 2, where data points for mometasone furoate once daily are shown by triangle symbols (▴), mometasone furoate twice daily data points are shown by square symbols (▪), and placebo data points are shown by diamond symbols (♦). From this graph, it is seen that those patients using the placebo inhalers generally declined in pulmonary function over the trial period, while the patients inhaling mometasone furoate particles generally improved their pulmonary function.

A statistical analysis of the data shows that the mean change from baseline over the twelve-month period was an increase in ΔFEV₁ of 50 mL for mometasone furoate dosed once daily, an increase in ΔFEV₁ of 53 mL for mometasone furoate dosed twice daily, and a decrease in ΔFEV₁ of 19 mL for placebo. Both treatments with mometasone furoate were statistically significantly superior to the placebo treatment.

What is claimed is:

1. A method for treating chronic obstructive pulmonary disease comprising administering by oral inhalation particles of mometasone furoate in daily doses where at least about 250 μg of the particles have sizes equal to or less than about 6.5 μm.

2. The method of claim 1, wherein less than about 50 μg of the mometasone furoate particles in a daily dose have sizes smaller than about 1 μm.

3. The method of claim 1, wherein the mometasone furoate is administered once daily.

4. The method of claim 3, wherein the mometasone furoate is administered in the evening.

5. The method of claim 1, wherein the daily dose of mometasone furoate is administered in two approximately equal portions, at intervals of about 12 hours.

6. The method of claim 1, wherein the daily dose comprises up to about 600 μg of mometasone furoate particles having sizes equal to or less than about 6.5 μm.

7. The method of claim 2, wherein a daily dose comprises at least about 200 μg of particles having diameters equal to or less than about 4.4 μm.

8. The method of claim 2, wherein a daily dose comprises at least about 175 μg of particles having diameters equal to or less than about 3.3 μm.

9. The method of claim 2, wherein a daily dose comprises at least about 75 μg of particles having diameters equal to or less than about 2 μm.

10. A method for treating chronic obstructive pulmonary disease comprising administering by oral inhalation particles of mometasone furoate in once-daily doses comprising up to about 600 μg of mometasone furoate particles having diameters equal to or less than about 6.5 μm.

11. The method of claim 10, wherein less than about 50 μg of mometasone furoate particles in a dose have diameters smaller than about 1 μm.

12. The method of claim 11, wherein a dose comprises at least about 200 μg of particles having diameters less than about 4.4 μm.

13. The method of claim 11, wherein a dose comprises at least about 175 μg of particles having diameters less than about 3.3 μm.

14. The method of claim 11, wherein a dose comprises at least about 75 μg of particles having diameters less than about 2 μm.

15. A method for treating chronic obstructive pulmonary disease comprising administering by oral inhalation particles of mometasone furoate in daily doses comprising up to about 600 μg of mometasone furoate particles having diameters equal to or less than about 6.5 μm, wherein at least about 200 μg of particles have diameters equal to or less than about 4.4 μm, at least about 175 μg of particles have diameters equal to or less than about 3.3 μm, at least about 75 μg of particles have diameters equal to or less than about 2 μm, and less than about 50 μg of mometasone furoate particles have diameters smaller than about 1 μm.

16. The method of claim 15, wherein the mometasone furoate is administered once daily.

17. The method of claim 16, wherein the dose is administered in the evening.

18. The method of claim 15, wherein the daily dose comprises at least about 250 μg of mometasone furoate particles having diameters equal to or less than about 6.5 μm.

19. A method for treating chronic obstructive pulmonary disease comprising administering by oral inhalation particles of mometasone furoate in a daily dose of 800 μg.

20. The method of claim 19, wherein the mometasone furoate is administered once daily.

21. The method of claim 20, wherein the mometasone furoate is administered in the evening.

22. The method of claim 20, wherein the mometasone furoate is administered to a patient who has a history of repeated COPD exacerbations.

23. The method of claim 21, wherein the mometasone furoate is administered to a patient who has a history of repeated COPD exacerbations.

24. The method of claim 20, wherein the chronic obstructive pulmonary disease is characterized by a moderate to very severe airway obstruction.

25. The method of claim 21, wherein the chronic obstructive pulmonary disease is characterized by a very severe airway obstruction.
